(19) United States
(12) Patent Application Publication
(10) Pub. No.: US 2016/0120912 A1
(43) Pub. Date: May 5, 2016

(54) COMPOSITIONS AND METHOD FOR PROMOTING NERVE GROWTH AND REGENERATION

(71) Applicant: TissueTech, Inc., Doral, FL (US)

(72) Inventor: Scheffer TSENG, Pinecrest, FL (US)

(21) Appl. No.: 14/933,106

(22) Filed: Nov. 5, 2015

Related U.S. Application Data
(60) Provisional application No. 62/075,444, filed on Nov. 5, 2014.

(51) Int. Cl. A61K 35/50; A61K 9/00; A61K 35/51

(52) U.S. Cl. CPC A61K 35/50; A61K 35/51; A61K 9/0048

(57) ABSTRACT

The invention relates generally to the fields of biology and health sciences. More particularly, the invention relates to compositions and methods for modulating cellular physiology and pathological processing using a combination of compounds that can be found in amniotic membrane tissue and umbilical cord tissue preparations.

### CROSS-REFERENCE TO RELATED APPLICATIONS

This application claims priority to U.S. Provisional Application No. 62/075,444 filed on Nov. 5, 2014 entitled “COMPOSITIONS AND METHOD FOR PROMOTING NERVE GROWTH AND REGENERATION,” of which is hereby incorporated by reference in its entirety.

### FIELD OF THE INVENTION

The invention relates generally to the fields of biology and health sciences. More particularly, the invention relates to compositions and methods for modulating cellular physiology and pathological processing using a combination of compounds that can be found in amniotic membrane tissue and umbilical cord tissue preparations.

### BACKGROUND

The cornea is the most densely innervated tissue in the body with a nerve density of 300-600 times the skin. These nerves play an important role in regulating corneal epithelial maintenance, tear production, and sensory function. Recently, data has been gathered showing the correlation between the loss of corneal nerve density and the severity of dry eye, suggesting sub-basal corneal nerves can be monitored as a way of gauging the severity and improvement of dry eye. Consequently, in vivo confocal microscopy (IVCM), a non-invasive imaging tool, has been used to monitor the nerves and quantitate their density, width, branching patterns, number of beads, tortuosity, reflectivity and/or orientation. Previous studies have also used IVCM to detect changes to ocular surface epithelium, immune and inflammatory cells, keratocytes, and stroma in dry eye patients. Most notably, a dramatic increase in epithelial dendritic cell density has been shown in dry eye patients compared to healthy controls and these corneal morphological characteristics have a direct relationship with corneal sensitivity. Collectively, these results demonstrate IVCM is a powerful platform that can be used to detect changes in the cornea and monitor the clinical efficacy of treatments for DED.

With each blink of the eyelids, tears are spread across the front surface of the eye, known as the cornea. Tears provide lubrication, reduce the risk of eye infection, wash away foreign matter in the eye, and keep the surface of the eyes smooth and clear. Excess tears in the eyes flow into small drainage ducts, in the inner corners of the eyelids, which drain in the back of the nose.

Dry eyes can result from an improper balance of tear production and drainage.

Tears are produced by several glands in and around the eyelids. Tear production tends to diminish with age, with various medical conditions, or as a side effect of certain medicines. Environmental conditions such as wind and dry climates can also affect tear volume by increasing tear evaporation. When the normal amount of tear production decreases or tears evaporate too quickly from the eyes, symptoms of dry eye can develop.

Tears are made up of three layers: oil, water, and mucus. Each component serves a function in protecting and nourishing the front surface of the eye. A smooth oil layer helps to prevent evaporation of the water layer, while the mucin layer functions in spreading the tears evenly over the surface of the eye. If the tears evaporate too quickly or do not spread evenly over the cornea due to deficiencies with any of the three tear layers, dry eye symptoms can develop.

The most common form of dry eyes is due to an inadequate amount of the water layer of tears. This condition, called keratoconjunctivitis sicca (KCS), is also referred to as dry eye syndrome.

People with dry eyes may experience symptoms of irritated, gritty, scratchy, or burning eyes, a feeling of something in their eyes, excess watering, and blurred vision due to nerve loss or nerve damage in the cornea. Advanced dry eyes may damage the front surface of the eye and impair vision.

Current treatments for dry eyes aim to restore or maintain the normal amount of tears in the eye to minimize dryness and related discomfort and to maintain eye health. What is needed is a treatment that can increase corneal sensation, increase nerve growth or regeneration and reduce the inflammatory response in patients suffering from dry eye.

### SUMMARY

In a first embodiment the present application describes a composition for promoting nerve growth, promoting nerve regeneration or a combination thereof, comprising at least one of: a.) a therapeutically effective amount of amniotic membrane tissue; and b.) a therapeutically effective amount of umbilical cord tissue. Additional embodiments exist, wherein the amniotic membrane tissue and the umbilical cord tissue may be present in any ratio from about 0.000:100.000 w/w % to about 100.000:0.000 w/w % of amniotic membrane tissue to umbilical cord tissue, respectively. Additional embodiments exist, wherein the composition comprises viable cells. Additional embodiments exist, wherein the composition is formulated to be a dosage form selected from the group consisting of: solid, ointment, cream, slurry, injectable solution, micronized powder, lyophilized solid and liquid. Additional embodiments exist, wherein the dosage form may be packaged in a container selected from the group consisting of: pouch, jar, bottle, tube, ampule and pre-filled syringe. Additional embodiments exist, wherein the natural biological activity of the amniotic membrane tissue and the umbilical cord tissue is substantially preserved for at least 15 days after initial procurement. Additional embodiments exist, wherein the composition increases corneal sensation. Additional embodiments exist, wherein the composition is anti-inflammatory when contacted with an exogenous living cell. Additional embodiments exist, wherein the composition is anti-inflammatory when contacted with an endogenous living cell. Additional embodiments exist, wherein substantially all red blood cells have been removed from the amniotic membrane tissue and the umbilical cord tissue. Additional embodiments exist, wherein substantially all chorion tissue has been removed from the amniotic membrane tissue and the umbilical cord tissue. Additional embodiments exist, wherein at least some chorion tissue remains with the amniotic membrane tissue and the umbilical cord tissue. Additional embodiments exist, wherein the composition also comprises amniotic fluid. Additional embodiments exist, wherein the composition is cryopreserved, lyophilized, dehydrated or a combination thereof. Additional embodiments exist, wherein the composition further comprises at least one pharmaceutically acceptable carrier or diluent selected from the group consisting of: acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, tricalcium phosphate, dipotassium phosphate, cellulose and cellulose conjugates, sugars sodium stearoyl lactylate, carrageenan, monoglyceride, diglyceride, pregelatinized starch, lactose, starch, mannitol, sorbitol, dextrose, microcrystalline cellulose, dibasic calcium phosphate, dicalcium phosphate dihydrate; tricalcium phosphate, calcium phosphate; anhydrous lactose, spray-dried lactose, compressible sugar, hydroxypropylmethylcellulose, hydroxypropylmethylcellulose acetate stearate, sucrose, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin, inositol and bentonite. Additional embodiments exist, wherein the composition further comprises at least one additional type of cell selected from the group consisting of: limbal epithelial stem cells, keratocytes, limbal stromal niche cells, human umbilical vein endothelial cells, mesenchymal stem cells, adipose-derived stem cells, endothelial stem cells and dental pulp stem cells. Additional embodiments exist, wherein the composition is a homogenate.

In another embodiment the present application describes a process for the preparation of a composition according to the application, comprising: a.) obtaining a therapeutically effective amount of amniotic membrane tissue selected from the group consisting of: fresh amniotic membrane tissue, frozen amniotic membrane tissue and a combination thereof; b.) obtaining a therapeutically effective amount of umbilical cord tissue selected from the group consisting of: fresh umbilical cord tissue, frozen umbilical cord tissue and a combination thereof; c.) mixing a therapeutically effective amount of amniotic membrane tissue with a therapeutically effective amount of umbilical cord tissue in any ratio from about 0.000:100.000 w/w % to about 100.00:0.000 w/w % of amniotic membrane tissue to umbilical cord tissue, respectively. Additional embodiments exist, wherein the mixing is accomplished with a tool selected from the group consisting of: tissue grinder, sonicator, bread beater, freezer/mill, blender, mortar and pestle, ruler and scalpel. Additional embodiments exist, wherein the process further comprises: d.) packaging the composition in a container selected from the group consisting of: pouch, jar, bottle, tube and ampule. Additional embodiments exist, wherein the natural biological activity of the isolated amniotic membrane tissue and the umbilical cord tissue is substantially preserved for at least 15 days after initial procurement. Additional embodiments exist, wherein the umbilical cord is obtained from a human, non-human primate, cow or pig. Additional embodiments exist, wherein the amniotic membrane tissue and the umbilical cord tissue composition promotes nerve growth, promotes nerve regeneration, promotes an anti-inflammatory response or a combination thereof when contacted with an exogenous living cell. Additional embodiments exist, wherein the amniotic membrane tissue and the umbilical cord tissue composition promotes nerve growth, promotes nerve regeneration, promotes an anti-inflammatory response or a combination thereof when contacted with an endogenous living cell. Additional embodiments exist, wherein the wherein the amniotic membrane tissue and the umbilical cord tissue are separated from substantially all the chorion tissue. Additional embodiments exist, wherein the amniotic membrane tissue and the umbilical cord tissue are separated from the umbilical vein and umbilical arteries and at least a portion of the Wharton's Jelly. Additional embodiments exist, wherein the process further comprises inhibiting the metabolic activity of substantially all cells found on the amniotic membrane tissue and the umbilical cord tissue by freezing or drying the umbilical cord. Additional embodiments exist, wherein the process further comprises draining blood from the umbilical cord before removing Wharton's Jelly, the umbilical vein, and the umbilical arteries. Additional embodiments exist, wherein the process further comprises removing substantially all red blood cells from the amniotic membrane tissue and the umbilical cord tissue. Additional embodiments exist, wherein the process further comprises lyophilizing, cryopreserving, or terminally sterilizing the amniotic membrane tissue and the umbilical cord tissue.

In another embodiment the present application describes a method for treating dry eye, wherein the method comprises: administering a therapeutically effective amount of a composition according to the application to a patient in need thereof.

In another embodiment the present application describes the use of the composition according to the application to promote an increase in tissue sensation.

In another embodiment the present application describes the use of a composition according to the application to induce a patient to blink and tear more frequently to prevent dry eye.

In another embodiment the present application describes the use of a composition according to the application to promote nerve growth, promote nerve regeneration or a combination in a contacted tissue. Additional embodiments exist, wherein the increase in nerve growth is between about 10% and about 100%. Additional embodiment exist, where the increase in nerve regeneration is between about 10% and about 100%.

In another embodiment the present application describe the use of a composition according to the application reduce an inflammatory response in a contacted tissue.

In another embodiment the present application describe the use of a composition according to the application to increase Tear Breakup Time in a patient suffering from dry eye disease.

In another embodiment the present application describe the use of a composition according to the application to increase tear osmolarity in a patient suffering from dry eye disease.

In another embodiment the present application describe the use of a composition according to the application to decrease corneal straining in a patient suffering from dry eye disease.

In another embodiment the present application describe the use of a composition according to the application to increase the score on Schirmer's test in a patient suffering from dry eye disease.

### BRIEF DESCRIPTION OF THE FIGURES

FIG. 1—Length of Time Suffering From Dry Eye.

FIG. 2—Previous Forms of Dry Eye Treatment.

FIG. 3—Patient Response After Treatment.

FIG. 4—Pain Relief Associated With Treatment.

FIG. 5—In vivo Confocal Microscopy of the Eye of a Patient Before and After One-Month Treatment With a Composition of the Present Application.

### DETAILED DESCRIPTION

The placenta is a temporary organ that surrounds the fetus during gestation. The placenta allows for transport of gases and nutrients, and also provides other metabolic and endocrine functions. The placenta is composed of several tissue types. The umbilical cord (UC) connects the placenta to the fetus, and transports oxygen to the fetus. The umbilical cord has two arteries and a vein. Wharton's jelly, a specialized gelatinous connective tissue material, is within the umbilical cord and protects and insulates the umbilical arteries and vein. The outermost layer of the amniotic sac is known as the “chorion.” Much of the placental disc is composed of chorionic villi, which are extensions of the chorionic villous tree. Through these structures, fetal nutrition exchange occurs. The amniotic membrane (AM) is an avascular membranous sac that is filled with amniotic fluid. This membrane is the innermost membrane surrounding a fetus in the amniotic cavity. This tissue consists of an epithelial layer and a subadjacent avascular stromal layer.

The umbilical cord (UC) and amniotic membrane (AM) are rich in stem cells and the resulting UCAM compositions will therefore meet an unfilled need in the field of dry eye treatment.

Although compositions, materials, and methods similar or equivalent to those described herein can be used in the practice or testing of the present invention, suitable preparations, methods and materials are described herein. All publications mentioned herein are incorporated by reference in their entirety. In the case of conflict, the present specification, including definitions will control. In addition, the particular embodiments discussed below are illustrative only and not intended to be limiting.

### Certain Definitions

The term “acceptable” with respect to a formulation, composition or ingredient, as used herein, means having no persistent detrimental effect on the general health of the subject being treated.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein, is an amount effective to achieve a desired effect or therapeutic improvement without undue adverse side effects. It is understood that “an effective amount” or “a therapeutically effective amount” can vary from subject to subject, due to variation in metabolism of the composition, age, weight, general condition of the subject, the condition being treated, the severity of the condition being treated, and the judgment of the prescribing physician.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “kit” and “article of manufacture” are used as synonyms.

By “pharmaceutically acceptable,” as used herein, refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutical combination” as used herein, means a product that results from the mixing or combining of more than one active ingredient and includes both fixed and non-fixed combinations of the active ingredients. The term “fixed combination” means that the active ingredients, e.g. the UCAM compositions described herein and a co-agent, are both administered to a patient simultaneously in the form of a single entity or dosage. The term “non-fixed combination” means that the active ingredients, e.g. the UCAM compositions described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “protein” as used herein can be the full length polypeptide, or a fragment or segment of a polypeptide, and can encompass a stretch of amino acid residues of at least about 8 amino acids, generally at least 10 amino acids, more generally at least 20 amino acids, often at least 30 amino acids, more often at least 50 amino acids or more of the full length polypeptide.

As used herein, the term “subject” is used to mean an animal, preferably a mammal, including a human or non-human. The terms patient and subject may be used interchangeably.

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Discussion

In an embodiment, the present invention describes compositions that are useful for promoting nerve growth, promoting nerve regeneration and a combination thereof. These compositions comprise at least one of amniotic membrane tissue, umbilical cord tissue or a combination thereof in any ratio from about 0.000:100.000 w/w % to about 100.000:0.000 w/w % of amniotic membrane tissue to umbilical cord tissue, respectively. The amniotic membrane tissue and the umbilical cord tissue may be present in the composition as particles of any size from about 0.1 mm to about 10.0 cm in length, width and thickness.

In a particular embodiment, the present invention describes a composition wherein the composition comprises UCAM tissue or AM tissue individually or UC tissue individually fastened onto a device or support, that may be, for example, in the shape of a conformer to be fitted to cover a portion of the corneal surface, the corneal surface, or the entire ocular surface. The support may be ring-shaped. The support with amniotic membrane attached thereto may be used as a temporary patch to increase corneal sensation, increase innervation and/or reduce inflammatory response in the contacted tissue, hence restoring comfort and vision.

In an additional embodiment, the present invention describes processes for the preparation of compositions useful for promoting nerve growth, promoting nerve regeneration and a combination thereof. These compositions comprise at least one of amniotic membrane tissue, umbilical cord tissue or a combination thereof in any ratio from about 0.000:100.000 w/w % to about 100.000:0.000 w/w % of amniotic membrane tissue to umbilical cord tissue, respectively. The amniotic membrane tissue and the umbilical cord tissue may be present in the composition as particles of any size from about 0.1 mm to about 3.0 cm in length, width and thickness.

### Results

The compositions of the present invention are useful for the treatment of dry eye. A composition of the present invention, comprising AM tissue fastened onto a ring-shaped support, in the shape of a conformer, to be fitted to cover the corneal surface was used in a clinical study to determine the efficacy of the compositions of the present invention at treating dry eye.

A clinical study to determine the efficacy of the compositions of the present invention was performed. The clinical study enrolled patients that suffered from dry eye and after treatment with the composition of the present invention they were surveyed to determine the results. One hundred and sixty (160) patients completed the treatment survey.

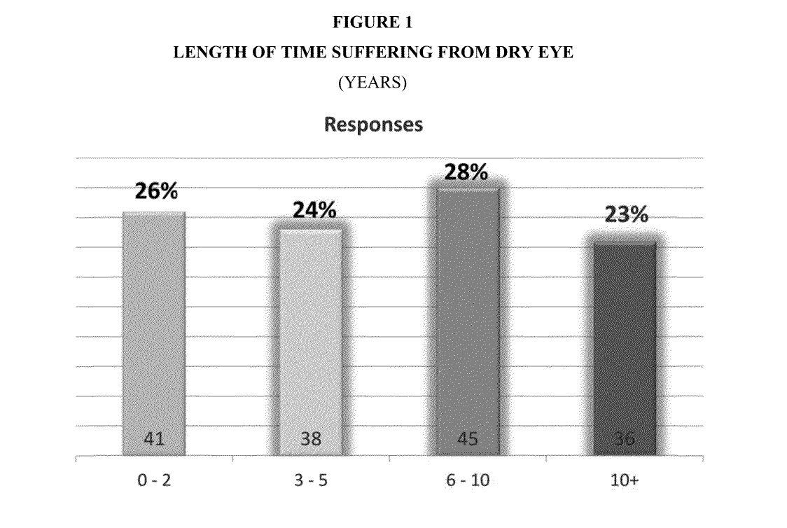

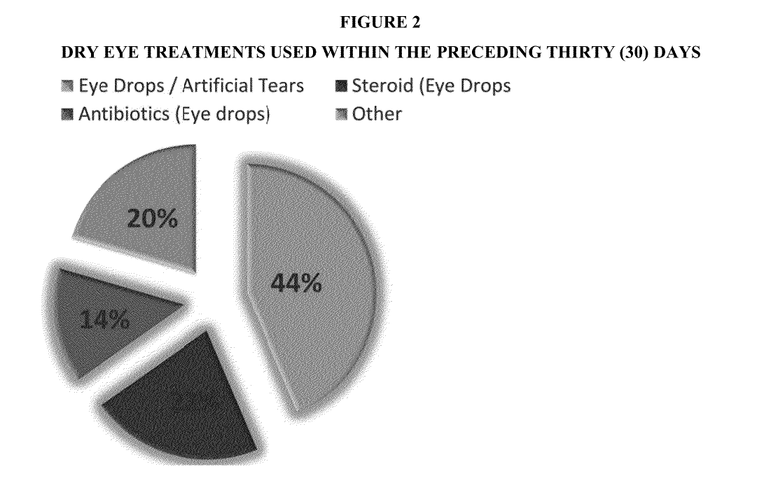

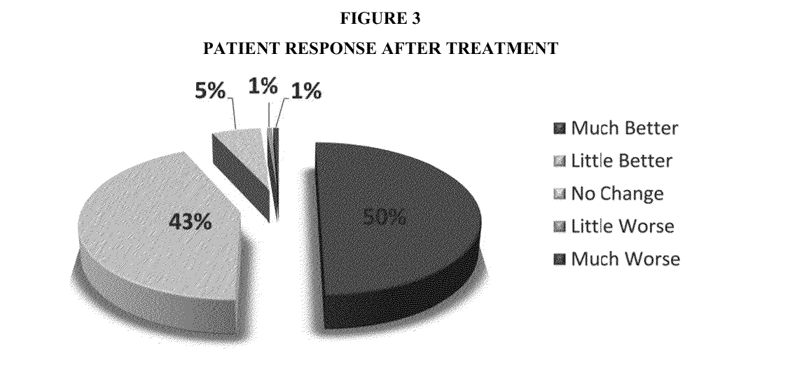

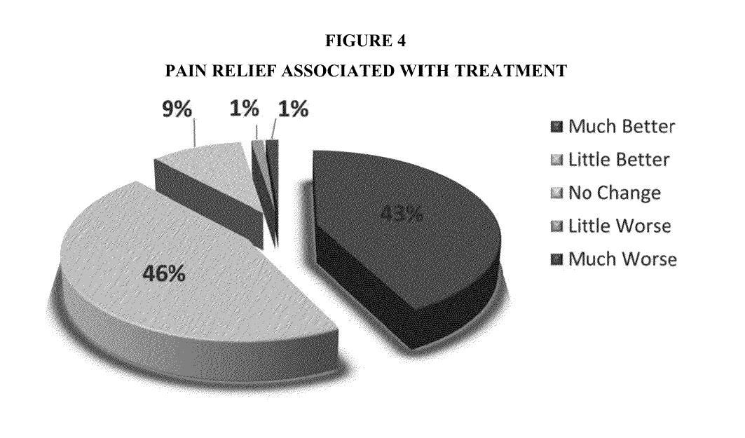

Sixty eight (68) percent of the patients were female and thirty two (32) percent of the patients were female and sixty six (66) percent of the patients were over 55 years in age. The patients selected how long they had been suffering from dry eye from the following categories 0-2 years; 3-5 years; 6-10 years; and 10+years. Seventy five (75) percent of the patients had suffered from dry eye for 3 years or more. (FIG. 1). Additionally, the patients selected what form of treatment they had been using to treat their dry eye, during the preceding thirty (30) days from the following categories eye drops/artificial tears; antibiotic eye drops; steroid eye drops; and other. (FIG. 2). Ninety three (93) percent of the patients said they felt better after treatment with the composition. (FIG. 3). Additionally, eighty nine (89) percent of the patients said the pain associated with dry eye was alleviated. (FIG. 4).

A second clinical study was performed to determine efficacy of the compositions of the present invention wherein twenty (20) patients, male or female, aged 21 years or older suffering from moderate to severe DED were enrolled. The patients had one eye treated with a formulation of the present invention as well as non-preserved artificial tears; whereas, the control eye only received non-preserved artificial tears. The patients were evaluated using IVCM during three (3) months follow-up. IVCM is a non-invasive method of examining the cornea in living humans and animals. It is especially valuable for evaluating the cornea nerves due to their important roles in regulating epithelial integrity, proliferation and wound healing. Patients suffering from dry eye demonstrate a loss of corneal innervation and the use of IVCM will allow for evaluation during treatment. IVCM can also be used to evaluate ocular surface epithelium, immune and inflammatory cells, dendritic cells, keratocytes, and stroma in dry eye patients.

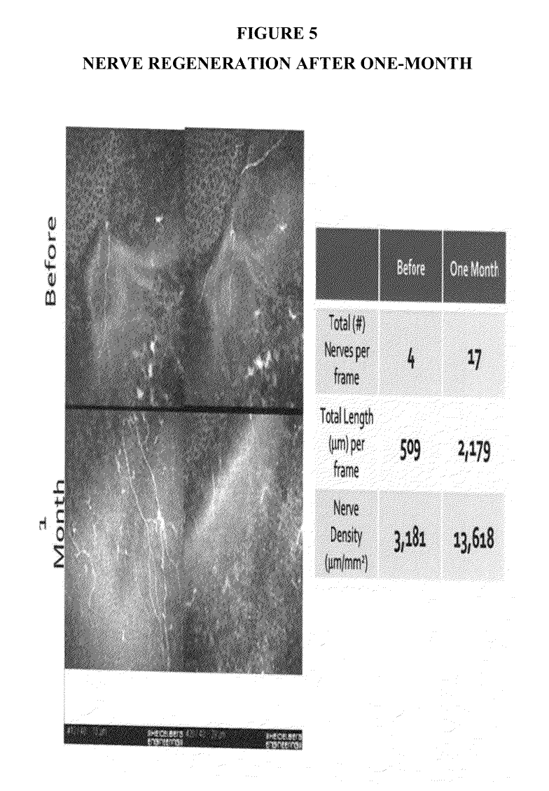

It was observed in one patient (FIG. 5) that the total number of nerves visible in one IVCM frame after one (1) month had increased from 4 to 17, the total nerve length had increased from 509 μm to 2,179 μm and the nerve density had increased from 3,181 μm/mm² to 13,618 μm/mm².

### Composition

Described herein are compositions that exert a number of physiologically significant effects in mammalian cells and intact mammalian tissues. The compositions comprise at least one of: amniotic membrane tissue and umbilical cord tissue.

Any or all of the components of the compositions described herein can be prepared from a human amniotic material, including human amniotic jelly preparations and extracts (as described herein), human amniotic membrane preparations and extracts (as described herein), and human amniotic stroma preparations and extracts (as described herein) or a human umbilical cord material (as described herein) including human Wharton's jelly preparations and extracts (as described herein).

These two components can suppress TGF β promoter activity; increase apoptosis in macrophages; decrease proliferation, decrease migration, and increase apoptosis of human vascular endothelial cells; decrease viability of human fibroblasts; decrease inflammation; and prevent apoptosis of epithelial cells exposed to storage and injury.

These components can be obtained from any suitable source. For example, at least one of the components can be obtained from human tissues, such as amniotic membrane, amniotic jelly, amniotic stroma, amniotic fluid, or a combination thereof. At least one of the components can be obtained from commercial sources. At least one of the components can be isolated from a transgenic organism. The protein sequences can have a similarity of at least 90%, 93%, 95%, 97%, 99% or 99.5% to the human protein sequence. The components can be purified, substantially purified, partially purified, or non-purified. The components can also be prepared from mammalian amniotic membrane tissues, as each of the components is present in amniotic membrane tissues.

Human placental material can be obtained, for example, from sources such as Bio-Tissue, Inc. (Miami, Fla.) and Baptist Hospital (Miami, Fla.) (under IRB approval). The tissue is typically obtained in either a fresh or frozen state. The tissue can be washed to remove excess storage buffer, blood, or contaminants. The excess liquid can be removed, for example, using a brief centrifugation step, or by other means. The tissue can be frozen, using, for example, liquid nitrogen or other cooling means, to facilitate the subsequent homogenization. The source of the UCAM tissue can be a human. However, other sources of UCAM tissue, such as bovine or porcine UCAM tissue, can be used.

A mixture of amniotic membrane tissue and umbilical cord tissue in any ratio from 0.001:99.999 w/w % to 99.999:0.001 w/w % can be prepared from either fresh or frozen tissue through the use of any tool known to one of skill in the art such as, for example, tissue grinder, sonicator, bread beater, freezer/mill, blender, mortar/pestle, Roto-stator, kitchen chopper, grater, ruler and scalpel to yield tissue ranging in size from about 0.1 mm to about 3.0 cm in length, width, or thickness. Optionally, the resulting tissue may be homogenized to yield consistently sized tissue. The resulting tissue may be either used wet, partially dehydrated or essentially dehydrated by any means known to one of skill in the art such as, for example, centrifuge or lyophilization. The resulting composition may be used immediately or stored for later use in any type of contained known to one of skill in the art such as, for example, pouch, jar, bottle, tube, ampule and pre-filled syringe. Finally, the composition may be sterilized by any method known to one of skill in the art such as, for example, γ radiation.

The placenta can be used to prepare the composition. UCAM preparations can include components or portions extracted from intact placentas. If desired, certain components of the UCAM preparation can be isolated from the preparation at any time during the process. The preparation can be dried, if desired.

The tissue can be frozen prior to the process. The freezing step can occur by any suitable cooling process. For example, the tissue can be flash-frozen using liquid nitrogen. Alternatively, the material can be placed in an isopropanol/dry ice bath or can be flash-frozen in other coolants. Commercially available quick freezing processes can be used. Additionally, the material can be placed in a freezer and allowed to equilibrate to the storage temperature more slowly, rather than being flash-frozen. The tissue can be stored at any desired temperature. For example, −20° C. or −80° C. or other temperatures can be used for storage.

Preparing the tissue while frozen, rather than preparing the tissue prior to freezing, is one optional method for preparing the tissue. Alternatively, fresh, partially thawed, or thawed tissue can be used. The tissue (fresh, frozen, or thawed) can then be sliced into pieces of a desired size with a suitable device, such as a scalpel, and homogenized with a homogenization device such as a laboratory blender, in a suitable solution. Exemplary solutions include but are not limited to phosphate buffered saline (PBS), DMEM, NaCl solution, and water. The pH of the solution can be adjusted as needed. In some embodiments, the pH range is from about 5.5 or 6.0 to about 8.5. In some embodiments, the frozen tissue is prepared in a solution having a pH of between about 6.3, about 6.6, or about 7.0 to about 7.4, about 7.6, or about 7.8.

UCAM preparations can be in a liquid, suspension, or lyophilized forms. Antimicrobial agents such as antibiotics or anti-fungal agents may be added. The material can be packaged and stored, for example, at room temperature, or for example, at −20° C. or −80° C. prior to use.

In some embodiments, the preparation is present as a dry formulation. A dry formulation can be stored in a smaller volume, and may not require the same low temperature storage requirements to keep the formulation from degrading over time. A dry formulation can be stored and reconstituted prior to use. The dry formulation can be prepared, for example, by preparing the freeze-morselized UCAM tissue as described herein, then removing at least a portion of the water in the composition. The excess water can be removed from the preparation by any suitable means. An exemplary method of removing the water is by use of lyophilization using a commercially available lyophilizer or freeze-dryer. Suitable equipment can be found, for example, through Virtis, Gardiner, N.Y.; FTS Systems, Stone Ridge, N.Y.; and SpeedVac (Savant Instruments Inc., Farmingdale, N.Y.). The amount of water that is removed can be from about 5%, 10%, 20%, 30% to about 60, 70, 80, 90, 95 or 99% or more. In some embodiments, substantially all of the excess water is removed. The lyophilized composition can then be stored. The storage temperature can vary from less than about −196° C. −80° C., −50° C., or −20° C. to more than about 23° C. If desired, the composition can be characterized (weight, protein content, etc.) prior to storage.

The lyophilized composition can be reconstituted in a suitable solution or buffer prior to use. Exemplary solutions include but are not limited to PBS, DMEM, and BSS. The pH of the solution can be adjusted as needed. The concentration of the UCAM can be varied as needed. In some procedures a more concentrated preparation is useful, whereas in other procedures, a solution with a low concentration of UCAM is useful. Additional compounds can be added to the composition. Exemplary compounds that can be added to the reconstituted formulation include but are not limited to pH modifiers, buffers, collagen, hyaluronic acid (HA), antibiotics, surfactants, stabilizers, proteins, and the like. The lyophilized UCAM composition can also be added to a prepared cream, ointment or lotion to result in the desired concentration.

The following procedures represent illustrative methods for preparing the UCAM compositions described and used herein.

### Preparation of Preserved Human UCAM

Human placenta was collected at elective cesarean delivery (Heiligenhaus et al., Invest Ophthalmol Vis Sci. 42:1969-1974, 2001, Lee and Tseng, Am J Ophthalmol. 123:303-312, 1997, Prabhasawat et al., Ophthalmology, 104:974-985, 1997, Tseng et al., Arch Ophthalmol. 116:431-441, 1998). The UCAM was flattened onto nitrocellulose paper (Hybond N+, Amersham, England), with the epithelium surface up. The UCAM samples were stored at −80° C. in DMEM/glycerol 1:2 (v/v) until use.

### UCAM Compositions

UCAM compositions can be formulated for administration purposes as a non-solid dosage form, for example, by combining with a delivery vehicle to create compositions such as solutions, drops, suspensions, pastes, sprays, ointments, oils, emulsions, aerosols, a coated bandage, a patch, creams, lotions, gels, and the like. The formulation used will depend upon the particular application. Gels are useful for administering the compositions because they allow better retention of the active ingredient at the site of introduction, allowing the active ingredient to exert its effect for a longer period of time before clearance of the active ingredient. A description of exemplary pharmaceutically acceptable carriers or vehicles and diluents, as well as pharmaceutical formulations, is provided herein and can also be found in Remington's Pharmaceutical Sciences, a standard text in this field, and in USP/NF.

Compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins; 1999), herein incorporated by reference in their entirety.

In certain embodiments, the compositions include a pharmaceutically acceptable diluent(s), excipient(s), or carrier(s). In addition, the UCAM compositions described herein can be administered as compositions in which UCAM compositions described herein are mixed with other active ingredients, as in combination therapy. In some embodiments, the compositions may include other medicinal or pharmaceutical agents, carriers, adjuvants, such as preserving, stabilizing, wetting or emulsifying agents, solution promoters, salts for regulating the osmotic pressure, and/or buffers. In addition, the compositions can also contain other therapeutically effective substances.

A composition, as used herein, refers to a mixture of a UCAM compositions described herein with other chemical components, such as carriers, stabilizers, diluents, dispersing agents, suspending agents, thickening agents, and/or excipients. The composition facilitates administration of the compound to an organism. In practicing the methods of treatment or use provided herein, therapeutically effective amounts of UCAM compositions described herein are administered to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

### Ophthalmic Formulations

Unless the intended purpose of use is affected adversely, the ophthalmic formulation of the present invention may further comprise one or more additional therapeutically-active agents. Specific therapeutically-active agents include, but are not limited to: antibacterial antibiotics, synthetic antibacterials, antifungal antibiotics, synthetic antifungals, antineoplastic agents, steroidal anti-inflammatory agents, non-steroidal anti-inflammatory agents, anti-allergic agents, glaucoma-treating agents, antiviral agents, and anti-mycotic agents. Further contemplated are any derivatives of the therapeutically-active agents which may include, but not be limited to: analogs, salts, esters, amines, amides, alcohols and acids derived from an agent of the invention and may be used in place of an agent itself

Examples of the antibacterial antibiotics include, but are not limited to: aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin sv, rifapentine, rifaximin), .beta.-lactams (e.g., carbacephems (e.g., loracarbef), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefmenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefmetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin g benethamine, penicillin g benzathine, penicillin g benzhydrylamine, penicillin g calcium, penicillin g hydrabamine, penicillin g potassium, penicillin g procaine, penicillin n, penicillin o, penicillin v, penicillin v benzathine, penicillin v hydrabamine, penimepicycline, phenethicillin potassium, piperacillin, pivampicillin, propicillin, quinacillin, sulbenicillin, sultamicillin, talampicillin, temocillin, ticarcillin), other (e.g., ritipenem), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, mikamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin s, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin).

Examples of the synthetic antibacterials include, but are not limited to: 2,4-diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benzylsulfamide, chloramine-b, chloramine-t, dichloramine t, N₂-formylsulfisomidine, N₄-β-d-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidocchrysoidine, sulfamoxole, sulfanilamide, 4-sulfanilamidosalicylic acid, N₄-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (e.g., clofoctol, hexedine, methenamine, methenamine anhydromethylene-citrate, methenamine hippurate, methenamine mandelate, methenamine sulfosalicylate, nitroxoline, taurolidine, xibornol).

Examples of the antifungal antibiotics include, but are not limited to: polyenes (e.g., amphotericin b, candicidin, dennostatin, filipin, fungichromin, hachimycin, hamycin, lucensomycin, mepartricin, natamycin, nystatin, pecilocin, perimycin), others (e.g., azaserine, griseofulvin, oligomycins, neomycin undecylenate, pyrrolnitrin, siccanin, tubercidin, viridin).

Examples of the synthetic antifungals include, but are not limited to: allylamines (e.g., butenafine, naftifine, terbinafine), imidazoles (e.g., bifonazole, butoconazole, chlordantoin, chlormiidazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole) others (e.g., acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, zinc propionate).

Examples of the antineoplastic agents include, but are not limited to: antineoplastc antibiotics and analogs (e.g., aclacinomycins, actinomycin anthramycin, azaserine, bleomycins, cactinomycin, carubicin, carzinophilin, chromomycins, dactinomycin, daunorubicin, 6-diazo-5-oxo-L-norleucine, doxorubicin, epirubicin, idarubicin, menogaril, mitomycins, mycophenolic acid, nogalamycin, olivomycines, peplomycin, pirarubicin, plicamycin, porfiromycin, puromycin, streptonigrin, streptozocin, tubercidin, zinostatin, zorubicin), antimetabolites exemplified by folic acid analogs (e.g., denopterin, edatrexate, methotrexate, piritrexim, pteropterin, TOMUDEX®, trimetrexate), purine analogs (e.g., cladribine, fludarabine, 6-mercaptopurine, thiamiprine, thioguanine), pyrimidine analogs (e.g., ancitabine, azacitidine, 6-azauridine, carmofur, cytarabine, doxifluridine, emitefur, enocitabine, floxuridine, fluorouracil, gemcitabine, tagafur).

Examples of the steroidal anti-inflammatory agents include, but are not limited to: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, and triamcinolone hexacetonide.

Examples of the non-steroidal anti-inflammatory agents include, but are not limited to: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (erg., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., am piroxicam, droxicam, isoxicam, lornoxicam, piroxicam, tenoxicam), E-acetamidocaproic acid, s-adenosylmethionine, 3-amino-4-hydroxybutyric acid, amixetrine, bendazac, benzydamine, .alpha.-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, and zileuton.

Examples of anti-allergic agents include, but are not limited to: tranilast, ketotifen fumarate, pheniramine, diphenhydramine hydrochloride, and sodium cromoglicate.

Examples of glaucoma-treating agents include, but are not limited to: pilocarpine hydrochloride, latanoprost, timolol, and isopropylunoprostone.

Examples of antiviral agents include, but are not limited to: idoxuridine, acyclovir, and trifluorouridine.

Examples of anti-mycotic agents include, but are not limited to: pimaricin, fluconazole, miconazole, amphotericin B, flucytosine, and itraconazole.

### Ophthalmic Combinations

Unless the intended purpose of use is affected adversely, the ophthalmic formulation of the present invention may be administered concurrently with one or more therapeutically-active agents. Specific therapeutically-active agents include, but are not limited to: antibacterial antibiotics, synthetic antibacterials, antifungal antibiotics, synthetic antifungals, antineoplastic agents, steroidal anti-inflammatory agents, non-steroidal anti-inflammatory agents, anti-allergic agents, glaucoma-treating agents, antiviral agents, and anti-mycotic agents. Further contemplated are any derivatives of the therapeutically-active agents which may include, but not be limited to: analogs, salts, esters, amines, amides, alcohols and acids derived from an agent of the invention and may be used in place of an agent itself.

Examples of the antibacterial antibiotics include, but are not limited to: aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin sv, rifapentine, rifaximin), .beta.-lactams (e.g., carbacephems (e.g., loracarbef), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefmenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefmetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin g benethamine, penicillin g benzathine, penicillin g benzhydrylamine, penicillin g calcium, penicillin g hydrabamine, penicillin g potassium, penicillin g procaine, penicillin n, penicillin o, penicillin v, penicillin v benzathine, penicillin v hydrabamine, penimepicycline, phenethicillin potassium, piperacillin, pivampicillin, propicillin, quinacillin, sulbenicillin, sultamicillin, talampicillin, temocillin, ticarcillin), other (e.g., ritipenem), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, mikamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin s, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin).

Examples of the synthetic antibacterials include, but are not limited to: 2,4-diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benzylsulfamide, chloramine-b, chloramine-t, dichloramine t, N₂-formylsulfisomidine, N₄-β-d-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidocchrysoidine, sulfamoxole, sulfanilamide, 4-sulfanilamidosalicylic acid, N₄-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (e.g., clofoctol, hexedine, methenamine, methenamine anhydromethylene-citrate, methenamine hippurate, methenamine mandelate, methenamine sulfosalicylate, nitroxoline, taurolidine, xibornol).

Examples of the antifungal antibiotics include, but are not limited to: polyenes (e.g., amphotericin b, candicidin, dennostatin, filipin, fungichromin, hachimycin, hamycin, lucensomycin, mepartricin, natamycin, nystatin, pecilocin, perimycin), others (e.g., azaserine, griseofulvin, oligomycins, neomycin undecylenate, pyrrolnitrin, siccanin, tubercidin, viridin).

Examples of the synthetic antifungals include, but are not limited to: allylamines (e.g., butenafine, naftifine, terbinafine), imidazoles (e.g., bifonazole, butoconazole, chlordantoin, chlormiidazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole) others (e.g., acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, zinc propionate).

Examples of the antineoplastic agents include, but are not limited to: antineoplastc antibiotics and analogs (e.g., aclacinomycins, actinomycin anthramycin, azaserine, bleomycins, cactinomycin, carubicin, carzinophilin, chromomycins, dactinomycin, daunorubicin, 6-diazo-5-oxo-L-norleucine, doxorubicin, epirubicin, idarubicin, menogaril, mitomycins, mycophenolic acid, nogalamycin, olivomycines, peplomycin, pirarubicin, plicamycin, porfiromycin, puromycin, streptonigrin, streptozocin, tubercidin, zinostatin, zorubicin), antimetabolites exemplified by folic acid analogs (e.g., denopterin, edatrexate, methotrexate, piritrexim, pteropterin, TOMUDEX®, trimetrexate), purine analogs (e.g., cladribine, fludarabine, 6-mercaptopurine, thiamiprine, thioguanine), pyrimidine analogs (e.g., ancitabine, azacitidine, 6-azauridine, carmofur, cytarabine, doxifluridine, emitefur, enocitabine, floxuridine, fluorouracil, gemcitabine, tagafur).

Examples of the steroidal anti-inflammatory agents include, but are not limited to: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, and triamcinolone hexacetonide.

Examples of the non-steroidal anti-inflammatory agents include, but are not limited to: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (erg., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., am piroxicam, droxicam, isoxicam, lornoxicam, piroxicam, tenoxicam), E-acetamidocaproic acid, s-adenosylmethionine, 3-amino-4-hydroxybutyric acid, amixetrine, bendazac, benzydamine, .alpha.-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, and zileuton.

Examples of anti-allergic agents include, but are not limited to: tranilast, ketotifen fumarate, pheniramine, diphenhydramine hydrochloride, and sodium cromoglicate.

Examples of glaucoma-treating agents include, but are not limited to: pilocarpine hydrochloride, latanoprost, timolol, and isopropylunoprostone.

Examples of antiviral agents include, but are not limited to: idoxuridine, acyclovir, and trifluorouridine.

Examples of anti-mycotic agents include, but are not limited to: pimaricin, fluconazole, miconazole, amphotericin B, flucytosine, and itraconazole.

### Viscosity/Osmolality/pH

The ophthalmic formulation when in an aqueous or non-aqueous form may also contain, but not be limited to: suspending agents (e.g., polyvinyl pyrrolidone, glycerin monostearate, sorbitan esters, lanolin alcohols) and dispersing agents (e.g., surfactants such as tyloxapol and polysorbate 80, ionic polymers such as sodium alginate) in addition to the agents listed above, to ensure that the ophthalmic formulation is satisfactorily dispersed in a uniform microparticulate suspension.

When the ophthalmic formulation is in the form of an aqueous suspension or solution, a non-aqueous suspension or solution, or a gel or ointment it is preferable to use a pH modifier to make the formulation have a pH between about 4 and 8, more preferably between about 6.8 to about 7.5. A preferred pH modifier is hydrochloric acid, sulfuric acid, boric acid, sodium hydroxide or any other ophthalmically-acceptable pH modifier.

According to a further aspect of the present invention a topical ophthalmically-acceptable formulation comprising physiologic levels of serum electrolytes in combination with a therapeutically-effective amount of an ophthalmically-active antimicrobial and an ophthalmically-active anti-inflammatory or steroidal agent to treat an ocular disease, injury or disorder may further comprise an ophthalmically-acceptable excipient which modulates the osmolality of the formulation from about 200 to about 500 mOsm/Kg, preferably from about 250 to about 400 mOsm/Kg, and more preferably from about 280 to about 320 mOsm/Kg.

Examples of osmolality excipients include, but are not limited to: dextrose, sodium chloride, potassium chloride, glycerin, various buffers and the like.

### Excipients

The formulation may contain various excipients incorporated ordinarily, such as buffering agents (e.g., phosphate buffers, borate buffers, citrate buffers, tartarate buffers, acetate buffers, amino acids, sodium acetate, sodium citrate and the like), isotonicity agents (e.g., saccharides such as sorbitol, glucose and mannitol, polyhydric alcohols such as glycerin, concentrated glycerin, polyethylene glycol and propylene glycol, salts such as sodium chloride), preservatives or antiseptics (e.g., benzalkonium chloride, benzethonium chloride, p-oxybenzoates such as methyl p-oxybenzoate or ethyl p-oxybenzoate, benzyl alcohol, phenethyl alcohol, sorbic acid or its salt, thimerosal, chlorobutanol, other quaternary amines and the like), solubilizing aids or stabilizing agents (e.g., cyclodextrins and their derivatives, water-soluble polymers such as polyvinyl pyrrolidone, or carbomer, surfactants such as polysorbate 80 (Tween 80)), pH modifiers (e.g., hydrochloric acid, acetic acid, phosphoric acid, sodium hydroxide, potassium hydroxide, ammonium hydroxide and the like), thickening agents (e.g., hydroxyethyl cellulose, hydroxypropyl cellulose, methyl cellulose, hydroxypropylmethyl cellulose, carboxymethyl cellulose and their salts), chelating agents (e.g., sodium edetate, sodium citrate, condensed sodium phosphate) and the like. Descriptions of compounds used in standard ophthalmic formulations may be found in, for example, Remington's Pharmaceutical Sciences, latest edition, Mack Publishing Co. Easton Pa.

Non-limiting examples of the contemplated excipients include a buffer, osmotic agent, demulcent, surfactant, emollient, tonicity agent, and/or a preservative component.

### Preparations

The formulation for ophthalmic conditions according to the present invention can be mixed with a ophthalmically acceptable carrier, excipient or diluent and formulated by a known method into a composition or formulation in various dosage forms such as injection solutions, eye drops and ophthalmic gels or ointments, and it is especially preferred to be used in a topical dosage form, preferably an eye drop formulation in solution or suspension form or an ophthalmic gel or ointment.

The ophthalmic formulation may for example be aqueous formulations such as aqueous eye drops, aqueous suspension eye drops, viscous eye drops and solubilized eye drops as well as non-aqueous formulations such as non-aqueous eye drops and non-aqueous suspension eye drops, or an ophthalmic gel or ointment.

The eye drop formulation in the form of an aqueous suspension preferably contains sodium citrate as a buffering agent, glycerin and/or propylene glycol as an isotonicity agent and polyvinyl pyrrolidone as a suspending agent.

The ophthalmic ointment may employ an ointment base known per se, such as purified lanolin, petrolatum, plastibase, liquid paraffin, polyethylene glycol and the like.

In another aspect of this invention, the ophthalmic formulation may be incorporated in a carrier system, which may be water, gel or ointment base. In still another aspect of this invention, said carrier system is a clear and stable pharmaceutical preparation, suitable for ocular treatment.

### Ophthalmic Inserts

In another aspect of the present invention, UCAM tissue or AM tissue individually or UC tissue individually is fastened onto a device or support, that may be, for example, in the shape of a conformer to be fitted to cover a portion of the corneal surface, the corneal surface, or the entire ocular surface. The support may be ring-shaped. The support with UCAM, AM or UC tissue attached thereto may be used as a temporary patch to increase corneal sensation, increase innervation and/or reduce inflammatory response in the contacted tissue, hence restoring comfort and vision.

In another aspect of the present invention, UCAM tissue or AM tissue individually or UC tissue individually is fastened on a device or support, that may be, for example, in the shape of a conformer to be fitted to cover a portion of the corneal surface, the corneal surface, or the entire ocular surface. The support may be ring-shaped.

A formulation of UCAM tissue or AM tissue individually or UC tissue individually is first applied to the cornea of a patient suffering from DED. Secondly, the support with UCAM, AM or UC tissue attached thereto may be used as a temporary patch to increase corneal sensation, increase innervation and/or reduce inflammatory response in the contacted tissue, hence restoring comfort and vision.

### EXAMPLES

### Example 1

Amniotic membrane tissue was obtained and flattened onto nitrocellulose paper, with the epithelium surface up. A surgical dermatome was used to prepare sheets of amniotic membrane tissue between about 50 μm and about 100 μm thick. The amniotic membrane tissue sheet were then cut to fit into a 15 mm internal diameter ring support that was designed to cover the cornea of a patient in need. The amniotic membrane tissue was mounted in the ring support such that the resulting ophthalmic device may be placed in the eye of a patient in need of treatment.

Although the present disclosure has been described in considerable detail with reference to certain preferred versions thereof, other versions are possible. Therefore, the spirit and scope of the application should not be limited to the description of the preferred versions described herein.

All features disclosed in the specification, including the abstract and drawings, and all the steps in any method or process disclosed, may be combined in any combination, except combinations where at least some of such features and/or steps are mutually exclusive. Each feature disclosed in the specification, including abstract and drawings, can be replaced by alternative features serving the same, equivalent or similar purpose, unless expressly stated otherwise. Thus, unless expressly stated otherwise, each feature disclosed is one example only of a generic series of equivalent or similar features. Various modifications of the application, in addition to those described herein, will be apparent to those skilled in the art from the foregoing description. Such modifications are also intended to fall within the scope of the appended claims.

What is claimed is:

1. A composition for promoting nerve growth, promoting nerve regeneration or a combination thereof, comprising at least one of:
a.) a therapeutically effective amount of amniotic membrane tissue; and
b.) a therapeutically effective amount of umbilical cord tissue.

2. The composition according to claim 1, wherein the amniotic membrane tissue and the umbilical cord tissue may be present in any ratio from about 0.000:100.000 w/w % to about 100.00:0.000 w/w % of amniotic membrane tissue to umbilical cord tissue, respectively.

3. The composition according to claim 1, wherein the composition comprises viable cells.

4. The composition according to claim 1, wherein the composition comprises non-viable cells.

5. The composition according to claim 1, wherein the composition is formulated to be a dosage form selected from the group consisting of: solid, ointment, cream, injectable solution, micronized powder, lyophilized solid, gel, slurry, and liquid.

6. The composition according to claim 5, wherein the dosage form may be packaged in a container selected from the group consisting of: pouch, jar, bottle, tube, ampule, eyedropper and pre-filled syringe.

7. The composition according claim 1, wherein the natural biological activity of the amniotic membrane tissue and the umbilical cord tissue is substantially preserved for at least 15 days after initial procurement.

8. The composition according to claim 1, wherein the composition further increases corneal sensation.

9. The composition according to claim 1, wherein the composition is anti-inflammatory when contacted with an exogenous living cell.

10. The composition according to claim 1, wherein the composition is anti-inflammatory when contacted with an endogenous living cell.

11. The composition according to claim 1, wherein substantially all red blood cells have been removed from the amniotic membrane tissue and the umbilical cord tissue.

12. The composition according to claim 1, wherein substantially all chorion tissue has been removed from the amniotic membrane tissue and the umbilical cord tissue.

13. The composition according to claim 1, wherein at least some chorion tissue remains with the amniotic membrane tissue and the umbilical cord tissue.

14. The composition according to claim 1, additionally comprising amniotic fluid.

15. The composition according to claim 1, wherein the composition is cryopreserved, lyophilized, dehydrated or a combination thereof.

16. The composition according to claim 1, wherein the composition further comprises at least one pharmaceutically acceptable carrier or diluent selected from the consisting of: water, phosphate buffered saline, polyethylene glycol, propylene glycol, mineral oil, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, tricalcium phosphate, dipotassium phosphate, cellulose and cellulose conjugates, sugars sodium stearoyl lactylate, carrageenan, monoglyceride, diglyceride, pregelatinized starch, lactose, starch, mannitol, sorbitol, dextrose, microcrystalline cellulose, dibasic calcium phosphate, dicalcium phosphate dihydrate; tricalcium phosphate, calcium phosphate; anhydrous lactose, spray-dried lactose, compressible sugar, hydroxypropylmethylcellulose, hydroxypropylmethylcellulose acetate stearate, sucrose, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; inositol and bentonite.

17. The composition according to claim 1, wherein the composition further comprises at least one additional type of cell selected from the group consisting of: limbal epithelial stem cells, limbal stromal niche cells, keratocytes, human umbilical vein endothelial cells, mesenchymal stem cells, adipose-derived stem cells, endothelial stem cells and dental pulp stem cells.

18. The composition according to claim 1, wherein the composition is a homogenate.

19. The composition according to claim 1, further comprising at least one additional therapeutic agent selected from the group consisting of: antibacterial antibiotics, synthetic antibacterials, antifungal antibiotics, synthetic antifungals, antineoplastic agents, steroidal anti-inflammatory agents, non-steroidal anti-inflammatory agents, anti-allergic agents, glaucoma-treating agents, antiviral agents, and anti-mycotic agents.

20. The composition according to claim 1, wherein the composition promotes nerve growth, promotes regeneration or a combination thereof in an exogenous tissue.

21. The composition according to claim 1, wherein the composition promotes nerve growth, promotes regeneration or a combination thereof in an endogenous tissue.

22. A process for the preparation of a composition according to claim 1, comprising:
a.) obtaining a therapeutically effective amount of amniotic membrane tissue selected from the group consisting of: fresh amniotic membrane tissue, frozen amniotic membrane tissue and a combination thereof;
b.) obtaining a therapeutically effective amount of umbilical cord tissue selected from the group consisting of: fresh amniotic umbilical cord tissue, frozen amniotic umbilical cord tissue and a combination thereof;
c.) mixing a therapeutically effective amount of amniotic membrane tissue with a therapeutically effective amount of umbilical cord tissue in any ratio from about 0.000:100.000 w/w % to about 100.000:0.000 w/w % of amniotic membrane tissue to umbilical cord tissue, respectively.

23. The process according to claim 22, wherein the mixing is accomplished with a tool selected from the group consisting of: tissue grinder, sonicator, bread beater, freezer/mill, blender, mortar and pestle, ruler and scalpel.

24. The process according to claim 22, wherein the process further comprises:
d.) packaging the composition in a container selected from the group consisting of: pouch, jar, bottle, tube, syringe, eyedropper and ampule.

25. The process according to claim 22, wherein the natural biological activity of the isolated amniotic membrane tissue and the umbilical cord tissue is substantially preserved for at least 15 days after initial procurement.

26. The process according to claim 22, wherein the umbilical cord is obtained from a human, non-human primate, cow or pig.

27. The process according to claim 22, wherein the amniotic membrane tissue and the umbilical cord tissue composition promotes nerve growth, promotes nerve regeneration, promotes an anti-inflammatory response or a combination thereof when contacted with an exogenous living cell.

28. The process according to claim 22, wherein the amniotic membrane tissue and the umbilical cord tissue composition promotes nerve growth, promotes nerve regeneration, promotes an anti-inflammatory response or a combination thereof when contacted with an endogenous living cell.

29. The process according to claim 22 wherein the wherein the amniotic membrane tissue and the umbilical cord tissue are separated from substantially all the chorion tissue.

30. The process according to claim 22, wherein the amniotic membrane tissue and the umbilical cord tissue are separated from the umbilical vein and umbilical arteries and at least a portion of the Wharton's Jelly.

31. The process according to claim 22, further comprising inhibiting the metabolic activity of substantially all cells found on the amniotic membrane tissue and the umbilical cord tissue by freezing or drying the umbilical cord.

32. The process according to claim 22, further comprising draining blood from the umbilical cord before removing Wharton's Jelly, the umbilical vein, and the umbilical arteries.

33. The process according to claim 22, further comprising removing substantially all red blood cells from the amniotic membrane tissue and the umbilical cord tissue.

34. The process according to claim 22, further comprising lyophilizing, cryopreserving, or terminally sterilizing the amniotic membrane tissue and the umbilical cord tissue.

35. A method for treating dry eye, wherein the method comprises: administering a therapeutically effective amount of a composition according to claim 1 to a patient in need thereof.

36. Use of the composition according to claim 1 to promote an increase in tissue sensation.

37. Use of a composition according to claim 1 to induce a patient to blink and tear more frequently to prevent dry eye.

38. Use of a composition according to claim 1 to promote nerve growth, promote nerve regeneration or a combination thereof in a contacted tissue.

39. The use according to claim 38, wherein the increase in nerve growth is between about 10% and about 100%.

40. The use according to claim 38, wherein the increase in nerve regeneration is between about 10% and about 100%.

41. Use of a composition according to claim 1 to reduce an inflammatory response in a contacted tissue.

42. Use of a composition according to claim 1 to increase Tear Breakup Time in a patient suffering from dry eye disease.

43. Use of a composition according to claim 1 to increase tear osmolarity in a patient suffering from dry eye disease.

44. Use of a composition according to claim 1 to decrease corneal straining in a patient suffering from dry eye disease.

45. Use of a composition according to claim 1 to increase the score on Schirmer's test in a patient suffering from dry eye disease.
